(19) United States
(12) Patent Application Publication
(10) Pub. No.: US 2010/0021401 A1
(43) Pub. Date: Jan. 28, 2010

(54) METHOD FOR TREATING SKIN

(76) Inventor: Liliane Sallander, Hasselby (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 12/444,686

(22) PCT Filed: Oct. 5, 2007
(86) PCT No.: PCT/US07/80501

§ 371 Date: Sep. 25, 2009

Related U.S. Application Data
(60) Provisional application No. 60/822,266, filed on Oct. 10, 2006.

(51) Int. Cl. A61K 38/39; A61Q 17/04; A61K 38/17; A61P 17/00

(52) U.S. Cl. 424/59; 514/8

(57) ABSTRACT

The method is for treating skin. A peptide is selected that affects a receptor so that the receptor is receptive to the peptide. The selected peptide is dissolved in a solution to form a peptide solution. The skin is vibrated to generate heat without reducing the peptide in the peptide solution. The peptide solution is applied to the skin. The peptide solution increases an operational activity of the receptor in the skin.

### TECHNICAL FIELD

The present invention relates to a method for treating skin. More particularly, the method is for treating the skin with a suitable amino acid such as a peptide compound.

### BACKGROUND OF THE INVENTION

Many attempts have been made to reduce or stop the aging effect on the skin. As the skin ages, wrinkles, lines, brown spots, grayish skin and puffiness are formed. The skin is composed of connective tissues. The skin also contains different cell types such as keratinocytes, melanocytes, fibroplast and Langerhans. The connective tissues are rich in extra cellular matrices (ECM) that are composed of four major groups including two fibrous molecules such as collagen and elastin and two glycoconjugates including glycoproteins and glycans, such as proteoglycans, glycosaminoglycans. Collagen is a protein that accounts for about 70% of the extra cellular matrix. Elastin only accounts for about 1-3% but is very important. Some of the components of the space between the fibers are filled mainly by proteoglycans and hyaluronan.

Glycoproteins such as fibronectin and laminin are involved in the matrix components. The dermal fibroplasts coordinate the biosynthesis of all the components of the extra cellular matrix. The aging effects on the extra cellular matrix include the loss of cells and ECM substance, undesirable modification of the skin thickness and the matrix composition. The aging of the skin also results in the loss of elastin that reduces the skin elasticity. There is also a loss of hydration due to loss of glycosaminoglycans.

Despite the many efforts to develop skin treatments, there is still no effective treatment of skin degradation. There is a need for a more effective way of treating skin to reduce or slow down undesirable changes to the skin.

### SUMMARY OF THE INVENTION

The method of the present invention provides a solution to the above-mentioned problems and drawbacks. More particularly, the method of the present invention is for treating skin. A peptide is selected that affects a receptor so that the receptor is receptive to the peptide. The selected peptide is dissolved in a solution to form a peptide solution. The skin is vibrated to generate heat without reducing the peptide in the peptide solution. The peptide solution is applied to the skin. The peptide solution increases an operational activity of the receptor in the skin.

### BRIEF DESCRIPTION OF THE DRAWING

FIG. 1 is a schematic cross-section of a skin segment; and

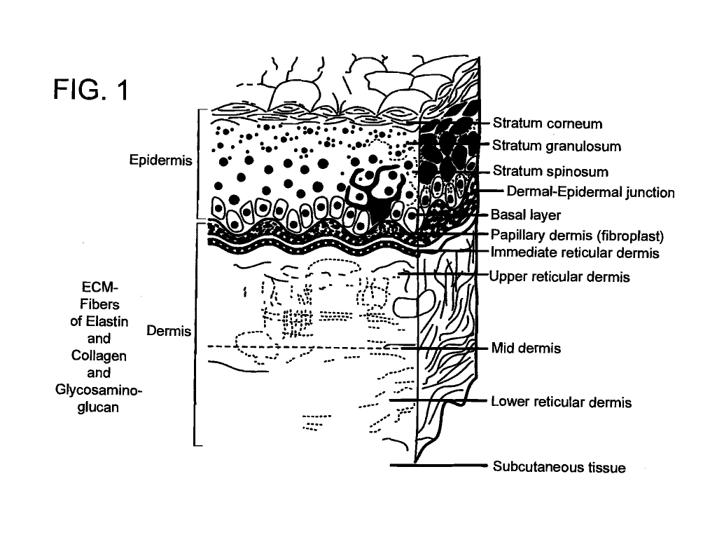

FIG. 2 is a schematic illustration showing receptors and the interaction between peptides and receptors.

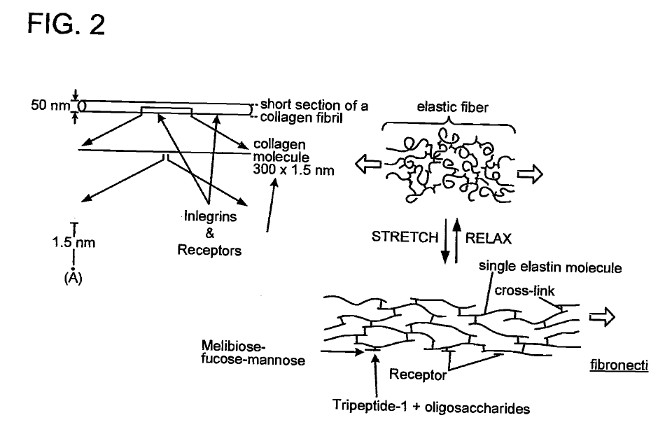

### DETAILED DESCRIPTION

An important aspect of the method of the present invention is that a suitable peptide is selected depending upon which receptor should be affected or stimulated. By changing the type of peptide used, different receptors will be affected. Another important and surprising discovery of the present invention is that the method pin-points exactly the parts of the skin that should be treated and preserved because short-chain amino acids, such as peptides, act only upon specific receptors, which in turn narrows down the natural reaction. To obtain the optimal effect it is of outmost importance to balance the peptides in specific and accurate compositions that affect the correct receptors and improve the overall skin.

For example, a surprising discovery is that tripeptides act upon and stimulate the elastin receptors together with melibiose and fucose-mannose catalysts disposed on the cell membranes and the added oligosaccharides (fucose) acts even upon the cell membranes.

Peptides are made of short chains of amino acids which are in different from proteins that have long chains of amino acids. In the body, peptides regulate the activity of other molecules such as proteins. This regulation is achieved by the interaction of the peptide with the target molecule i.e. the molecule to be treated. Peptides have many functions and some can have hormonal activity and other antibiotic functions.

As indicated above, the dermis contains fibroplasts which synthesize an interstitial tissue such as ECM that is composed of several compounds. ECM includes collagen, with two main proteins (I & III) which make up the system of fibers that gives the skin its strength. It also includes elastin, a protein fiber with a molecular structure that allows for stretching and tension, and makes the skin supple. ECM further includes laminin, a binding protein located at the junction of the dermis and epidermis, which acts in the interaction of the cell/matrix. It also includes fibronectin, which belongs to the same category and is involved in the healing and homeostasis of the skin.

Deterioration of the mechanical properties of ageing skin is a consequence of ECM atrophy. In order to prevent premature ageing of the skin it is of outmost importance to maintain the integrity of its matrix.

It has been discovered that peptides may be used to repair skin defects. More particularly, it was surprisingly discovered that a cream that contains tripeptide-1 participates in tissue repair and restarts extra cellular matrix renewal. Fibroplastine contributes to the repair of micro-injuries to the skin. It regenerates the skin that has been damaged from normal ageing or the environment due to damage to the dermis.

More particularly, it was surprisingly discovered that peptide could be combined with a suitable natural yeast/sugar substance to further reduce the degradation process. An important feature of the method of the present invention is to directly affect the receptors.

There are several receptors involved in the communications between the skin cells and the ECM. Integrins are specific receptors that affect the aging process of the skin. Elastic receptor with laminin receptors may be directly disturbed due to age-related factors and therefore release more damaging free radicals. This leads to a degradation of elastin. Additionally, fibronectin degradation is directly linked to elastin degradation.

More particularly, about 10 ppm tripeptide-1 in a liter of a solution of 10% oligosaccharides, that may be derived from a yeast such as saccharomyces cerevisiae and fucose-mannose, may be used to reduce or even prevent the degradation of fibronectine. The composition also induces the syntheses of collagen protein and the production of fibronectin that in turn produces elastin. The composition also induces the production of laminin which is an amino acid that builds up fibronectin.

The oligosaccharides solution is mainly used because it protects and strengthens the cell membrane that in turn protects the receptors that are located at and around the cell membrane. When the cell membrane is damaged then the receptors are often damaged too. By applying a composition of the tripeptide-1 and saccharides solution, the user may see an improvement of the skin in just a couple of days. It may be possible to use from about 0.1 ppm tripeptide-1 to up to about 20 ppm tripeptide-1. Preferably, about 7-13 ppm tripeptide-1 should be used. It may also be possible to use 1% oligosaccharides and up to 30% oligosaccharides solution. Preferably, between about 5-15% of oligosaccharides solution should be used.

A peptide composition of the present invention may also be used for protecting the skin against discoloration and from harmful sun light. Melanin is a pigment that gives the color of the skin. The amount of melanin produced is the basis of the skin pigmentation. Melanocytes are the cells that are producing melanin and are located at the basal layer of the skin. Melanotropin (or alpha-MSH) is the hormone that stimulates the melanocytes to produce melanin. The main purpose of melanin is to protect the skin from the dangers of UVA/UVB. The melanin pigment is the most natural protection of the skin. The natural color of the skin is the indication of the level of the skin's own natural defense and depends upon the type of melanin, eumelanin or phaeomelanin and the quantity of melanin that exists in the skin. Melanin is the substance that has the broadest UV spectrum and, both by its own action and during its synthesis, it consumes free radicals presented in the cells.

Melanin is synthesized from one amino acid called tyrosine. It is then oxidized to DOPA (DihydrOxy-Phenyl-Alanine) and then to Dopaquinone due to the action of a specific enzyme called tyrosinase. In this way, two different melanin substances may be produced. Melanin is synthesized inside the melanosomes located inside the melanocytes.

The melanosomes are transported into the keratinocytes (skin cells) where melanin is delivered directly inside the skin cells. While inside the skin cells the melanin may be totally degraded in some humans such as white and red haired humans. These complex syntheses may not only explain the mechanism of photo protection but also the process related to age spots or brown spots.

When hyperactivity of melanocytes occurs, melanin production becomes out of control. This may be due to over-exposure to sun, age, genetic factors, etc. The results are increased skin pigmentation in some part of the skin and remain the same in other parts of the skin due to malfunctioning of the proper natural degradation. Therefore, age spots or brown spots are visible.

It was surprisingly discovered that a combination of peptide and carotene provides an excellent protection against harmful sunlight by ensuring a steady regular and evenly distributed production of melanin.

More particularly, a solution of 0.05 mg/ml of acetyl hexapeptide-1 blended into 0.1 mg/ml beta-carotene solution is the base on a sun protection cream of the present invention. The acetyl hexapeptide-1 ensures that the right amount of melanin is produced so that not too much melanin is produced that creates dark spots. It also prevents too little melanin from being produced in the skin which is harmful due to reduced protection against sun light.

The above composition is based upon the acetyl hexapeptide-1 which is a biomimetic peptide. When this peptide is applied topically it stimulates melanogenesis but does not have an overall systemic effect. The composition also acts on the melanocyte by stimulating the MC-1r receptor and inducing endocellular synthesis of tyrosinase. Langerhatine raises the level of permanent protection against UV. Its natural stimulation action on melanogenesis makes the composition the ideal sun preparation product and suntan prolonging care.

As indicated above, peptides may also be used to treat spots on the skin. Sun exposure without protection will inevitably produce brown spots or age spots on the skin. These spots are named lentigines and increase in number with age. This phenomenon is explained as a result of a hyper activity of the melanocytes that abnormally synthesize melanin. The melanotropine (alpha-MSH) biological factor of melanogenese stimulation, acts by reacting upon a specific receptor located on the surface of the melanocyte. The peptide nonapeptide-1 used is an antagonist biomimetic reverse peptide which blocks the alpha-MSH and its effects. Moreover, by acting only upon a receptor located only on the membrane of the cell, melanosine does not influence any cell activity thus its physiological function.

As explained above, the ECM is the interstitial tissue that connects the cells and structures the skin. In the dermis, it is composed of collagen fibers associated with elastin fibers and this combination gives the skin its strength and suppleness. With age, repeated exposures to the sun lead to troubles collectively known as actinic ageing, one of the effects of which is elastosis or flabbiness. This is characterized by an accumulation of degraded elastin in the dermis.

The enzymes responsible for this breakdown are proteases, often metallo-proteases. These include elastases, the cellular production of which is strongly increased by exposure to solar radiation. It was surprisingly discovered that peptides may also be used to reduce the elastosis. More particularly, the peptide trifluoracetyl tripeptide-2 is a biomimetic peptide that inhibits elastases with a great accuracy. By inhibiting the breakdown of elastin and cutaneous proteins, keratosine efficiently combats the premature ageing of the skin and balance the adverse effects of sun exposure.

To preserve the skin from pollution, UVA/UVB and weather, the skin forms it s own protective barrier than can be disturbed due to the use of inappropriate skin care products. This barrier is located primarily in the outmost dermis layer. The dermis is built with skin cells called keratinocytes. Not only do these cells protect the skin but they are also of utmost important as a communication link with the underlying epidermis where among other things the immune cells, such as Langerhans cells, are located. Therefore, the healthy keratinocytes are basic for a healthy young looking skin.

Keratinocytes also have the ability to glue together the dermis to the epidermis. From the age of about thirty, our keratinocytes start losing their ability to stick to the dermo-epidermal junction. The degradation of keratinocytes leads to a weakening of this characteristic. This is one of the factors that communications between the dermis and epidermis do not function optimally. As a result, the skin looses its firmness and become grayish since the cells lose contact with blood capillaries that nourish them. Therefore, age factors and sun exposure are slowing down the renewal effectiveness of keratinocytes that leads to a degradation of the skin appearance.

More particularly, keratinocytes produce keratins, the mechanical properties of which provide the skin's first physical barrier. By secreting intercellular communication factor, these same cells also condition the environmental balance of the other components of the epidermis, including Langerhans cells which provide the first immunological barrier. The slowing down of keratinocytes renewal that occurs as skin ages disturbs both the physical and immunological barriers.

It was surprisingly discovered that peptides acts directly on the keratinocytes. More particularly, acetyl tetrapeptide-2 acts directly upon the cytokine of the keratinocytes. An effective composition is acetyl tetrapeptide-2 at a concentration of 0.01 mg/ml of serilesine and a solution of synthetic hexa-peptide from laminin at a concentration of 0.05 mg/ml are blended into a cream to repair age degradation of the skin. Serilesine is included in laminin amino acids that are used for building cells. The acetyl tetrapeptide-2 regulates the cytokine hormone secretion so that the cell building is indirectly affected by affecting the hormones of the cell.

The cytokine is a growth hormone that not only stimulates keratinocytes but also Langerhans cells. Because the cytokine hormone affects the production of Langerhans cells, it is useful as a day cream when the skin needs a good immune system the most. In this way, both the production of the immune cells and the cell building are stimulated at the same time.

The peptide, acetyl tetrapeptide-2, is a biomimetic of the cell maturation factors produced by the thymus. Oxytosine acts topically on the keratinocytes, inducing endocellular synthesis of paracrine and autocrone mediators. Oxytosine then stimulates the production of GM-CSF (Granulocyte Macrophage-Colony Stimulating Factor) by the keratinocytes. This cytokine stimulates not only keratinocytes renewal but also Langerhans cell maturation and improves the epidermis profile and stimulates the skin immune system.

The peptide system of the present invention may also be used on the skin around the eyes. The skin around the eyes is very delicate. It is the thinnest area of the body. This is also where the first lines appear. Furthermore, poor blood circulation in this area results in poor skin condition caused by less intake of oxygen. Adding UV exposure further leads to wrinkles and puffiness. To reverse these factors, an eye cream should have a primary function of awakening the natural micro-circulation and provide a renewal in oxygenation of the particular area without irritating the area.

It was surprisingly discovered that peptide in combination with a soy protein are effective. More particularly, 0.10 mg/ml lymphocyte activating peptide (Cas 120484-65-3) blended into a solution of 5% hydrolyzed rice bran protein glycine soy (soybean) protein oxido-reductase is very effective. One important function of the lymph substances is to discharge waste products from the skin. When the lymph substances are not functioning well it is common to swell and get puffy eyes. The stimulation of the lymph flow is therefor desirable. The lymphocyte activating peptide provides this stimulation. The soybean refines, softens and opens up the lymph passages and capillaries to make it easier for the lymph to flow in the skin area around the eyes. About 0.05-0.15 mg/ml lymphocyte activating peptide is preferably used in the above-described soy bean solution of between 3-10%.

The method use of applying the peptide on the skin is important. On a perfectly cleansed skin, a small amount of the peptide composition is applied. The composition is upwardly vibrated in order to induce the peptides into the skin. The vibrations are very important due to the fact that vibrations produce a slight heating reaction and allow the peptides to quickly penetrate into the skin without being reduced. Peptides are often very active and sometimes volatile and should be applied right away to maintain the effectiveness.

Furthermore, vibrations are generating a slight decollation or transverse movement of the epidermis that is necessary in order to preserve the proper quality of the peptides when applied. It is undesirable to apply a pressure on the skin to deform the skin because that makes the penetration of the peptides more difficult. The entire procedure of application should not last more than a few seconds. In the eye area when vibrations are difficult to achieve, light tapping on the skin is used to apply the peptide-based eye cream. Under no circumstances should usual massage be used in order to apply the products since this deforms the skin and does not induce the proper penetration process.

While the present invention has been described in accordance with preferred compositions and embodiments, it is to be understood that certain substitutions and alterations may be made thereto without departing from the spirit and scope of the following claims.

1. A method of treating skin, comprising: selecting a peptide that affects a receptor so that the receptor is receptive to the peptide; dissolving the selected peptide a solution to form a peptide solution; vibrating the skin to generate heat without reducing the peptide in the peptide solution; applying the peptide solution to the skin; and the peptide solution increasing an operational activity of the receptor in the skin.

2. The method according to claim 1 wherein the method further comprises providing a tripeptide substance to affect elastin receptors.

3. The method according to claim 2 wherein the method further comprises providing a tripeptide solution containing between 7-13 ppm tripeptide in an oligosaccharides solution.

4. The method according to claim 3 wherein the method further comprises using the tripeptide solution to reduce a degradation of fibronectine and induce a synthesis of a collagen protein and stimulate a production of fibronectin and laminin.

5. The method according to claim 1 wherein the method further comprises providing an acetyl tetrapeptide-2 at a concentration of 0.01 mg/ml of serilesine to regulate a cytokine hormone secretion.

6. The method according to claim 1 wherein the method further comprises providing a solution of acetyl hexapeptide-1 into a beta-carotene solution to provide sun protection.

7. The method according to claim 1 wherein the method further comprises providing a lymphocyte activating penta-peptide blended in a protein solution to stimulate a micro-circulation in the skin.
